(19) United States
(12) Patent Application Publication
(10) Pub. No.: US 2011/0151529 A1
(43) Pub. Date: Jun. 23, 2011

(54) METHODS AND PROCESSES FOR PRODUCING ORGANIC ACIDS

(75) Inventor: Shang-Tian Yang, Dublin, OH (US)

(73) Assignee: THE OHIO STATE UNIVERSITY, Columbus, OH (US)

(21) Appl. No.: 12/999,196

(22) PCT Filed: Jun. 19, 2008
(86) PCT No.: PCT/US08/67457

§ 371 Date: Feb. 9, 2011

(51) Int. Cl. C12P 7/52; C12P 7/40

(52) U.S. Cl. 435/141; 435/136

(57) ABSTRACT

A fermentation process for the production of organic acids is provided and includes, providing metabolically engineered mutant bacteria which have had one or both of ack and pta genes disrupted, adapting the mutant bacteria to increase their resistance to acids and to increase their growth rate by immobilizing the mutant bacteria while exposing the mutant bacteria to a fermentable substrate, and further exposing the adapted mutant bacteria to a fermentable substrate for a time sufficient to provide a final organic acid fermentation product concentration of greater than about 50 g/L.

The present invention is generally directed to methods for producing organic acids, and more specifically to methods of producing butyric acid, propionic acid, and acetic acid via fermentation using acid-tolerant strains of anaerobic bacteria derived from metabolic and process engineering.

Concerns about the future scarcity, cost, and environmental impact of fossil fuel have stimulated interest in the exploitation of cheap, renewable biomass as alternative sources for fuels and chemicals. As crude oil prices have risen, biobased chemicals and industrial products have become attractive alternatives to the petroleum-derived counterparts. Fermentation processes using anaerobic microorganisms provide a promising path for converting biomass and agricultural wastes into chemicals and fuels. There are abundant low-value agricultural commodities and food processing byproducts/wastes that require proper disposal to avoid pollution problems. These biomass waste materials can be used as low-cost feedstock for production of fuels and chemicals such as organic acids.

Production of butyric acid and hydrogen from renewable resources has become an increasingly attractive alternative to petroleum-based processes because of the increasing costs for petroleum-based raw materials, public concerns about the environmental pollution caused by the petrochemical industry, and consumers' preference for bio-based natural ingredients in foods, cosmetics, and pharmaceuticals. Butyric acid is a short-chain fatty acid produced from sugars by clostridial species such as C. tyrobutyricum, C. butyricum, C. beijerinckii, C. acetobutyricum, C. populeti, and C. thermobutyricum. Although butyric acid is currently produced mainly by petrochemical routes, there is increasing interest in butyric acid production from natural resources by fermentation. However, conventional fermentation technologies for butyric acid production are limited by low reactor productivity, product concentration, and yield because the butyrate-producing bacteria are heterofermentative and strongly inhibited by butyric acid.

Similarly, conventional propionic acid fermentation processes suffer from low productivity, yield, and final product concentration and purity, and are not economical for commercial applications. Propionic acid is an important chemical widely used in the production of cellulose plastics, herbicides, and perfumes. Propionic acid is also an important mold inhibitor. Its ammonia, calcium, sodium, and potassium salts are widely used as food and feed preservatives. Currently, the majority of propionic acid sold commercially is produced via petrochemical processes. However, there has been increasing interest in producing propionic acid from fermentation of sugars by propionibacteria. Although there have been some successes in improving fermentation productivity and final product concentration through various process and strain improvements, how to further improve propionic acid yield and purity in the fermentation process remains a challenge.

The development of an economically viable fermentation process for chemical production requires a microorganism that not only has high rates of production but also can tolerate high concentrations of the fermentation product. Methods for the improvement of industrial microorganisms range from the random approach of classical strain improvement (CSI) to the highly rational methods of metabolic engineering. Although CSI is robust, it is time and resource intensive. To obtain strains with high tolerance to inhibitory fermentation products, continuous screening and selection of mutants by successively culturing in the media with increasing inhibitor concentrations is usually used in conjunction with inducing mutagenesis by chemical mutagens or UV radiation. However, the conventional culture screening process is tedious, time-consuming, and often fruitless. More recently, recombinant DNA technology has also been applied to improve cell tolerance to inhibitory products. These methods are generally more effective, but are also more complicated to use and require the knowledge of detailed inhibition mechanism at the genetic level, which is not available for most of the microorganisms with industrial fermentation interests.

Metabolic engineering is widely used to design engineered strains to achieve higher efficiencies in metabolite overproduction through alterations in the metabolic flux distribution. Most metabolic engineering work to date is related to the production of secondary metabolites (such as antibiotics), amino acids (e.g., lysine), and heterologous proteins using organisms with well studied genetics and physiology (e.g., E. coli, yeast, and hybridoma cells). Stoichiometric analysis of metabolic flux distributions provides a guide to metabolic engineering, optimal medium formulation and feeding strategies, and bioprocess optimization, which, however, requires in-depth knowledge of the metabolic and regulatory networks in the fermentation cells. Although rational metabolic engineering approach has been successful in cases involving single gene or a few genes within a gene cluster, it has been ineffective in many cases involving complex or largely unknown metabolic pathways. This is because rational metabolic engineering approach usually targets one gene at a time and thus fails to predict complex interactions among multiple genes in the pathway.

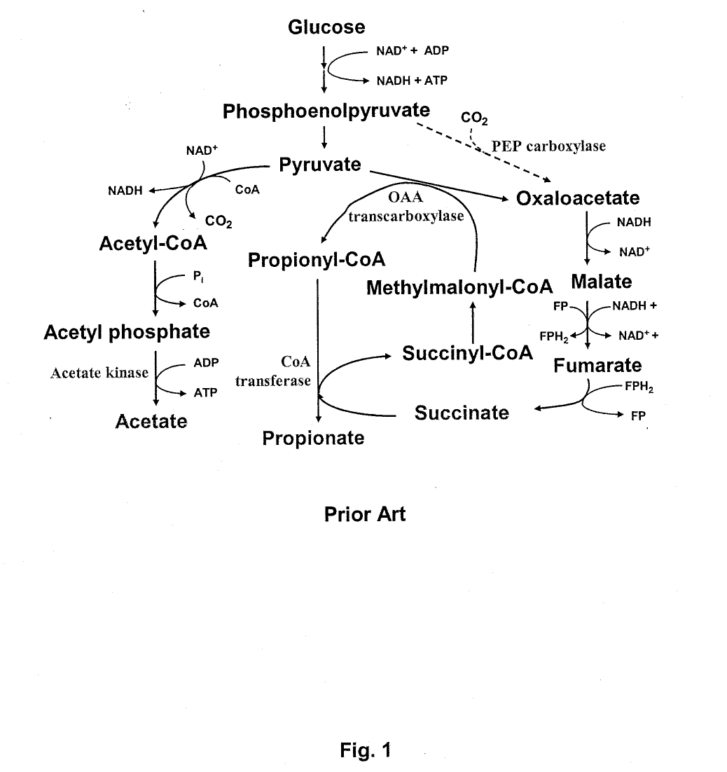

Propionibacteria are Gram-positive, catalase-positive, nonspore-forming, nonmotile, facultative anaerobic, rod-shaped bacteria. Members of the genus Propionibacteria are widely used in the production of vitamin B₁₂, tetrapyrrole compounds, and propionic acid, as well as in probiotic and cheese industries. As shown in FIG. 1, propionic acid is produced from the dicarboxylic acid pathway by propionibacteria and is usually accompanied by the formation of acetate and carbon dioxide. Theoretically, one mol glucose produces only 4/3 mol propionate and 2/3 mol acetate when glycolysis is through EMP (Embden-Meyerhof-Parnas) pathway. The actual propionate yield is much lower when there is significant cell growth and biomass formation. Even at a relatively low concentration of 10 g/L, propionate is a strong inhibitor to the fermentation. Typical batch propionic acid fermentation takes ˜3 days to reach ˜20 g/L propionic acid, with a propionic acid yield is usually less than 0.4 g/g glucose. Attempts to improve the propionic acid fermentation in terms of its yield, final product concentration and production rate have resulted in the development of new bioprocesses and mutant strains but with limited success. Significant improvements in the fermentation process are needed in order to make biobased propionic acid economically competitive with its petroleum counterparts and other related chemicals.

Clostridium tyrobutyricum is a gram-positive, rod-shaped, spore-forming, obligate anaerobic bacterium capable of fermenting a wide variety of carbohydrates to butyric and acetic acids. Historically, butyric acid fermentation in cheese (late blowing) caused by the outgrowth of Clostridial spores present in raw milk, most commonly originating from silage, can result in considerable product loss. On the other hand, butyric acid has many applications in chemical, food, and pharmaceutical industries. It is used in the form of pure acid to enhance butter-like notes in food flavors. Esters of this acid are used as additives for increasing fruit fragrance and as aromatic compounds for the production of perfumes.

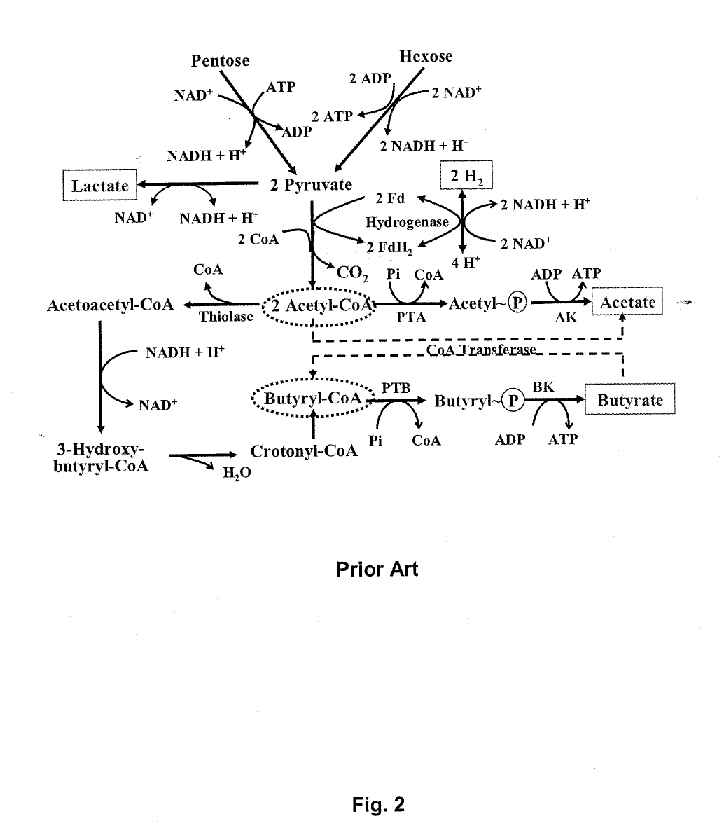

Butyric acid is one of the short-chain fatty acids generated by bacterial fermentation of dietary fibers in the colon, and has been shown to have anticancer effect and can be used as neutraceuticals or even as a drug to cure colorectal cancers. The fermentation pathway for butyric acid production from sugars is shown in FIG. 2. Acetic acid and hydrogen are also produced in the fermentation. Several anaerobic bacteria can produce butyric acid as the major fermentation product from a wide range of substrates. Among them, C. tyrobutyricum has many advantages over other species, including simple medium for cell growth and relatively high product purity and yield.

However, like other acidogens, butyric acid bacteria are strongly inhibited by their acid products. Consequently, conventional butyric acid fermentation is usually limited by low reactor productivity (<0.5 g/L·h), low product yield (<0.4 g/g), and low final product concentration (<30 g/L), making product recovery difficult and the process uneconomical. Many studies have been directed towards increasing cell density, reactor productivity and final butyric acid concentration, but only with limited success. Also, butyric acid is not the sole fermentation product. Acetic acid is also produced as a byproduct, which not only reduces butyric acid yield, but also makes the final product recovery more difficult and challenging.

One common problem in propionic acid and butyric acid fermentations is the co-production of acetate, which not only lowers the yield of the main fermentation product, but also causes difficulty in product purification. The propionic acid and butyric acid yields from glucose can be greatly increased if the substrate carbon is redistributed via metabolic engineering. For example, stoichiometric analysis of the propionic acid fermentation pathway indicates that the percentage of glucose catabolized through the HMP (hexose monophosphate) and/or EMP pathways has profound effects on propionate production, acetate production, and adenosine triphosphate (ATP) generation, and generally more propionate can be produced if more glucose is directed through the HMP pathway and acetate production is reduced. This is, however, at the expense of less ATP generation. The bacteria naturally would not take this route because of the energy consideration, but can be forced to do so if pyruvate oxidation to acetate is blocked.

The propionate yield can be further enhanced if CO₂ generated in the HMP pathway can be re-incorporated into the dicarboxylic acid pathway through phosphoenolpyruvate (PEP) carboxylase. Similarly, the production of acetate in the butyric acid fermentation plays a key role in redox balance and ATP generation, but can be reduced by redirecting the carbon flux towards the butyrate production pathway. Therefore, to inactivate genes in the acetate formation pathway is a viable method for enhancing propionate and butyrate yields in these anaerobic fermentations.

An integrational mutagenesis technique was developed to disrupt acetate kinase (ack) and phosphotransacetylase (pta) genes in C. tyrobutyricum and P. acidipropionici. See, Y. Zhu, X. Liu and S. T. Yang, Construction and Characterization of pta Gene Deleted Mutant of Clostridium tyrobutyricum for Butyric Acid Fermentation, Biotechnol. Bioeng., 90:154-166 (2005); X. Liu, Y. Zhu and S. T. Yang, Construction and Characterization of ack Deleted Mutant of Clostridium tyrobutyricum for Enhanced Butyric Acid and Hydrogen Production, Biotechnol. Prog., 22(5):1265-1275 (2006); and S. Suwannakham, Y. Huang and S. T. Yang, Construction and Characterization of ack Knock-out Mutants of Propionibacterium acidipropionici for Enhanced Propionic Acid Fermentation, Biotechnol. Bioeng., 94(2): 383-395 (2006). Non-replicative integrational plasmid constructs, containing ack or pta gene fragments and an antibiotic resistance cassette, were introduced into the cells, and inactivation of ack or pta occurred as a result of the integration of the plasmid into the homologous regions on the chromosome.

Consequently, the final concentration and productivity of butyrate and propionate increased, but cell growth rate decreased significantly as expected in the respective mutants as compared to their wild types. Final product concentrations were still less than about 50 g/L and below the threshold for economical commercial-scale recovery and purification. The reduced cell growth rate of the mutant bacteria would also limit their application in conventional fermentation processes. The gene knock out increased cell tolerance to butyrate and propionate of the respective mutant, but did not eliminate acetate production.

One major limitation in organic acid fermentations is the low final product concentration caused by product inhibition (low acid tolerance of the bacteria). How to enhance cellular tolerance to toxic by-products has been a major issue in many fermentation processes. However, some problems associated with conventional propionic acid and butyric acid fermentations (and many other carboxylic acid fermentations), including low acid tolerance, can be partially addressed by cell immobilization. In Yang, U.S. Pat. No. 5,563,069, a fibrous-bed immobilized-cell bioreactor (FBB) was developed for several organic acid fermentations which provided improved productivity, yield, and product concentration.

Using the FBB resulted in final product concentrations that were 2 to 3-times higher than those in conventional free-cell fermentation processes, not only because of the higher cell density in the FBB, but also through the adaptation of the culture to be more tolerant to the fermentation product. This process engineering approach through spontaneous adaptation and mutation in the fibrous bed bioreactor provides a way to obtain mutant strains with high acid tolerance and fermentation ability suitable for industrial production of organic acids from biomass. The FBB also makes possible the production of propionate and butyrate at high fermentation rate and high yield using slow-growth or non-growing immobilized cells. See, Y. Zhu and S. T. Yang, Enhancing Butyric Acid Production with Mutants of Clostridium tyrobutyricum Obtained from Metabolic Engineering and Adaptation in a Fibrous-Bed Bioreactor, in B. C. Saha (ed.), “Fermentation Biotechnology,” ACS Symposium Series No. 862, American Chemical Society (2003), pp. 52-66Y; Zhu, Z. Wu and S. T. Yang, Butyric Acid Production from Acid Hydrolysate of Corn Fiber by Clostridium tyrobutyricum in a Fibrous Bed Bioreactor, Process Biochemistry, 38:657-666 (2002); Y. Zhu and S. T. Yang, Adaptation of Clostridium tyrobutyricum for Enhanced Tolerance to Butyric Acid in a Fibrous-Bed Bioreactor, Biotechnol. Progress, 19:365-372 (2003); and S. Suwannakham and S.-T. Yang. Enhanced Propionic Acid Fermentation by Propionibacterium acidipropionici Mutant Obtained by Adaptation in a Fibrous-Bed Bioreactor, Biotechnol. Bioeng., 91:325-337. (2005). Again, however, final product concentrations were still less than about 50 g/L and below the threshold for economical commercial-scale recovery and purification.

Accordingly, the need still exists for methods for producing organic acids, and more specifically to methods of producing butyric acid, propionic acid, and acetic acid via fermentation using anaerobic bacteria at growth rates, yields, and product concentrations which will permit economical commercial-scale recovery and purification of the organic acid products.

Embodiments of the present invention meet that need by providing methods for producing organic acids, and more specifically to methods of producing butyric acid, propionic acid, and acetic acid via fermentation using anaerobic bacteria at growth rates, yields, and product concentrations which will permit economical commercial-scale recovery and purification of the organic acid products.

In accordance with one embodiment, a fermentation process for the production of organic acids is provided and comprises providing metabolically engineered mutant bacteria which have had one or both of ack and pta genes disrupted, adapting the mutant bacteria to increase their resistance to acids and to increase their growth rate by immobilizing the mutant bacteria while exposing the mutant bacteria to a fermentable substrate, and further exposing the adapted mutant bacteria to a fermentable substrate for a time sufficient to provide a final organic acid fermentation product concentration of greater than about 50 g/L.

In one embodiment, the mutant bacteria comprise C. tyrobutyricum PAK-Em and the organic acid fermentation product comprises butyric acid and hydrogen. The fermentable substrate comprises a sugar or other carbon substrate. In a preferred form, the sugar is selected from the group consisting of glucose, fructose, xylose, lactose, maltose, sucrose, and mixtures thereof. The carbon substrate may be selected from sources such as lactate and glycerol.

In another embodiment, the mutant bacteria comprise C. tyrobutyricum HydEm and the organic acid fermentation product comprises butyric acid and hydrogen. The fermentable substrate comprises a sugar.

In another embodiment, the mutant bacteria comprise C. tyrobutyricum PPTA-Em and the organic acid fermentation product comprises butyric acid and hydrogen. The fermentable substrate comprises a sugar.

In yet another embodiment, the mutant bacteria comprise P. acidipropionici ACK-Tet and the organic acid fermentation product comprises propionic acid. The fermentable substrate comprises a sugar. Alternatively, the fermentable substrate comprises glycerol.

In yet another embodiment, the mutant bacteria comprise P. acidipropionici TAT-ACK-Tet and the organic acid fermentation product comprises propionic acid. The fermentable substrate comprises a sugar. Alternatively, the fermentable substrate comprises glycerol.

By using a combination of metabolic and process engineering techniques, acid-tolerant bacterial strains have been developed for fermentation processes to produce organic acids at growth rates, product yields, and final product concentrations that were previously unachievable using other methods. The final product concentration exceeds the threshold required for economical recovery and purification of organic acid products, including propionic and butyric acids. In preferred embodiments, final product concentrations of greater than 80 g/L are achieved, permitting economical recovery and purification from the fermentation broth.

This combination of metabolic and process engineering dramatically increases the acid tolerance of the bacterial strains which cannot be achieved using only immobilized wild-type bacterial strains or by genetic engineering alone. Only with the combination of techniques does it become possible to induce the bacterial strains methodologically in a stimulating environment in a fibrous bed bioreactor to become extremely tolerant to the organic acid products of fermentation. Such a result is unpredictable based on previous knowledge in the art.

In accordance with embodiments of the present invention, a fibrous-bed bioreactor (FBB) with bacterial cells immobilized in a fibrous matrix may be used for the production of organic acids, including acetic, propionic, and butyric acids. With high cell densities immobilized in the fibrous matrix of the reactor, productivity, final product concentration, and product yield are all increased as compared to conventional free-cell fermentation processes. Immobilizing the metabolically engineered strains in a fibrous bed reactor provides an environment for the cells to quickly adapt and provide enriched cultures with a high tolerance to inhibitory fermentation products. This results in an increase in the final product concentration above a threshold level where economical recovery and purification of the organic acids in the fermentation broth is possible.

In a preferred embodiment, metabolic engineering is utilized to at least partially disrupt the acetate formation pathway in bacteria to further enhance butyric acid and propionic acid production by Clostridium tyrobutyricum and Propionibacterium acidipropionici, respectively. Acetate kinase (ack) and/or phosphotransacylase (pta) genes on the bacterial genome are partially inactivated (i.e., disrupted) to reduce acetate production by the bacteria. The mutations result in increased production of butyric acid by C. tyrobutyricum and propionic acid by P. acidipropionici, respectively.

Lowered growth rates due to reduced acetate and energy production of the metabolically engineered bacterial strains is addressed by immobilizing the engineered cells and adapting them in a fibrous bed reactor to improve and restore cell growth rates and improve fermentation performance. The spontaneous adaptation and mutation of the bacterial strains by immobilization in a fibrous bed bioreactor provides the to obtain mutant strains suitable for production of organic acids, including butyric acid and propionic acid, from sugars and other biomass on an industrial scale. The sequential adaptation of metabolically engineered mutant bacterial strains in a fibrous bed reactor provides an economical and efficient process for organic acid production from biomass. These and other features and advantages of embodiments of the invention will become apparent from the following detailed description, the accompanying drawings, and the appended claims.

FIG. 1. (Prior Art) depicts the dicarboxylic acid pathway for propionic acid fermentation by Propionibacterium acidipropionici.

FIG. 2 (Prior Art) depicts the metabolic pathway for butyric acid fermentation by Clostridium tyrobutyricum.

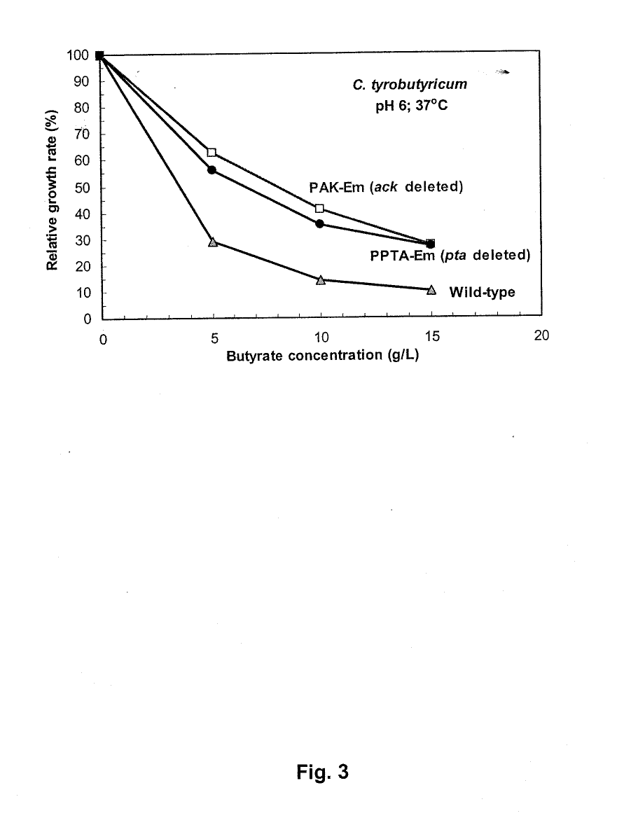

FIG. 3 is a chart of relative growth rate (%) versus butyrate concentration that shows the inhibition effects of butyric acid on cell growth of C. tyrobutyricum wild type, mutant PAK-Em, and mutant PPTA-Em. The relative specific growth rate with the one at zero butyrate concentration being 100% was used here for easier comparison between the wild type and the mutants.

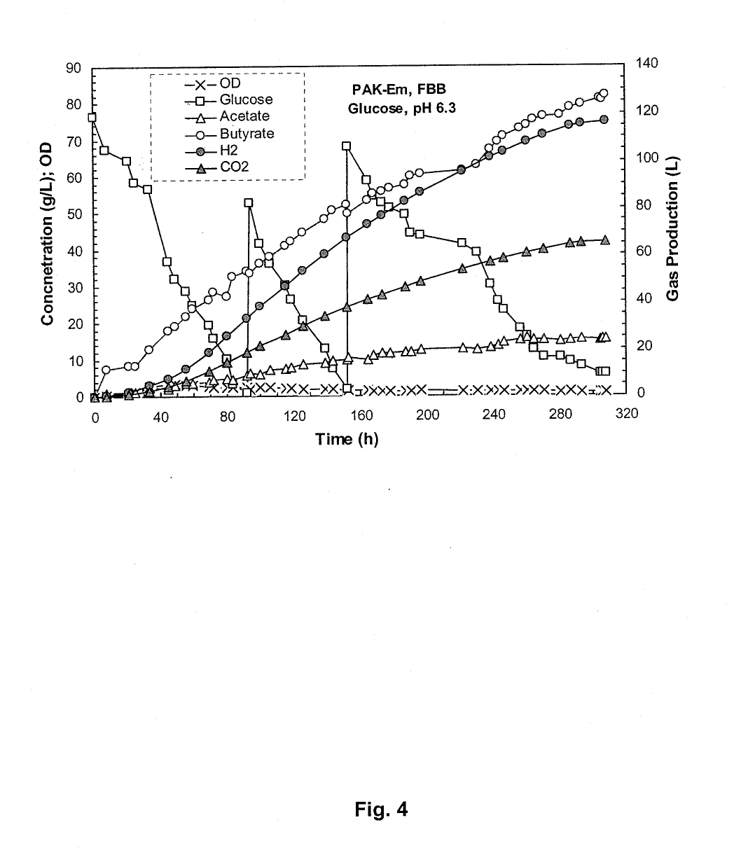

FIG. 4. is a chart of concentration (g/L) versus time (h) for a fermentation by immobilized cells of C. tyrobutyricum mutant PAK-Em in a fibrous bed reactor at 37° C. and pH 6.3 with glucose as the substrate.

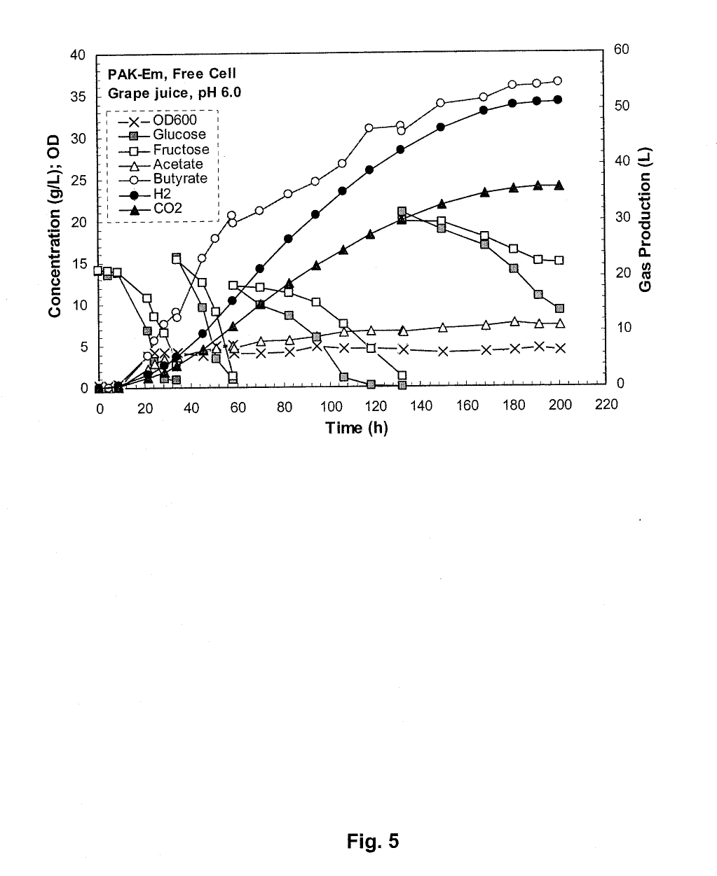

FIG. 5 is a chart of concentration (g/L) versus time (h) for a fermentation by free cells of C. tyrobutyricum mutant PAK-Em at 37° C. and pH 6.0 with grape juice containing glucose and fructose as the substrates.

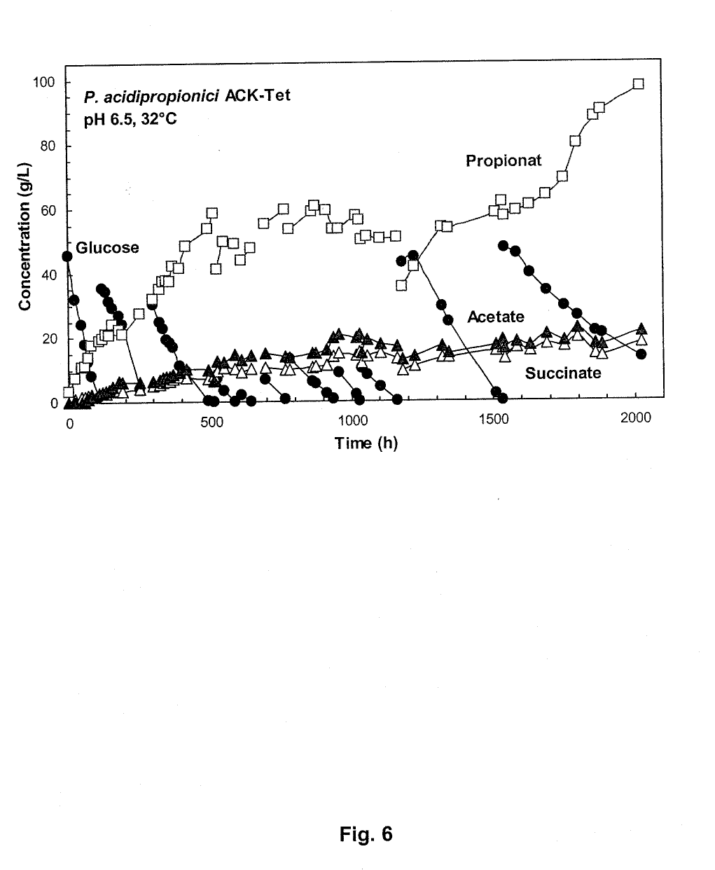

FIG. 6 is a chart of concentration (g/L) versus time (h) for a long-term propionic acid fermentation of glucose with P. acidipropionici ACK-Tet cells immobilized in a fibrous bed reactor at pH 6.5, 32° C.

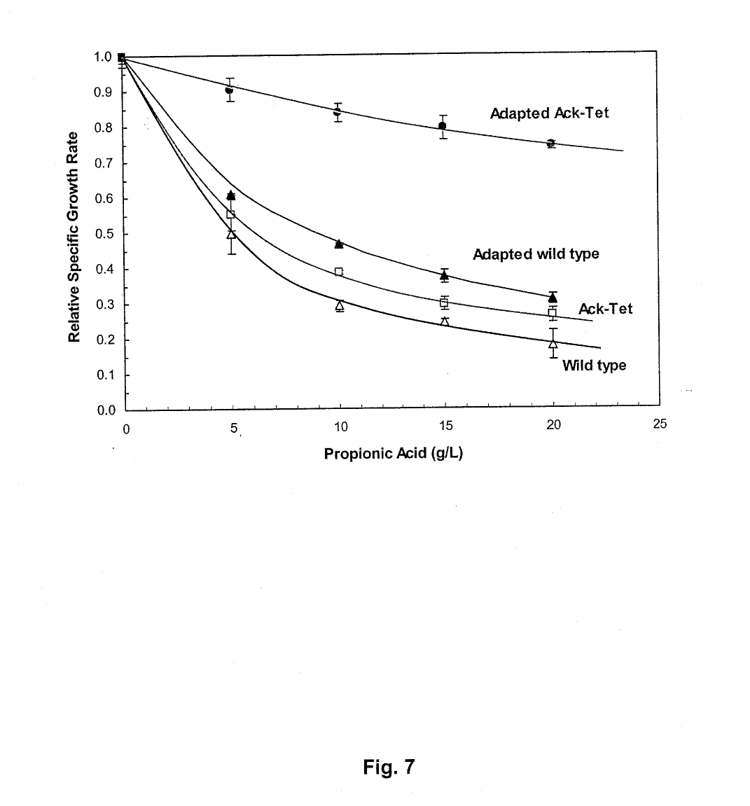

FIG. 7 is a chart of relative specific growth rate versus propionic acid concentration (g/L) for various strains of P. acidipropionici in free-cell culture at pH 6.5, 32° C.

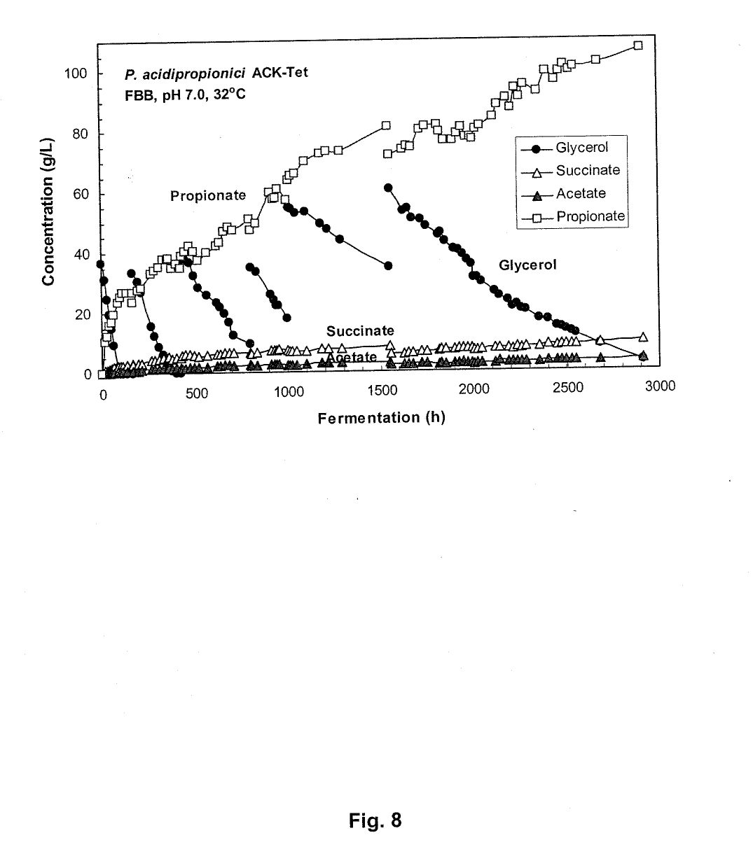

FIG. 8 is a chart of concentration (g/L) versus fermentation time (h) for a long-term propionic acid fermentation of glycerol with P. acidipropionici ACK-Tet cells immobilized in a FBB at pH 7.0, 32° C.

### BUTYRIC ACID FERMENTATION

Many butyric acid bacteria, including Clostridium tyrobutyricum, produce butyrate, acetate, H₂ and CO₂ as primary fermentation products from lactate and carbohydrate substrates, including glucose and xylose derived from starchy and lignocellulosic materials. To improve the fermentation process for butyric acid production, a sequential combination of metabolic engineering and process engineering techniques were applied to develop mutant bacterial strains with high acid tolerance and the ability to produce greater amounts of butyric acid and hydrogen from glucose and xylose.

Metabolic Engineering of Clostridium tyrobutyricum

As shown in FIG. 1, the metabolic pathways for butyrate and acetate production in C. tyrobutyricum and C. acetobutyricum have been extensively studied. In general, glucose is catabolized via the EMP (Embden-Meyerhof-Parnas) pathway and xylose is catabolized via the HMP (hexose monophosphate) pathway to pyruvate. Pyruvate is then oxidized to acetyl-CoA and carbon dioxide with concomitant reduction of ferredoxin (Fd) to FdH₂, which is then oxidized by hydrogenase to Fd, producing hydrogen and converting NAD⁺ to NADH. Acetyl-CoA is a key metabolic intermediate at the node dividing the acetate-forming branch, catalyzed by phosphotransacetylase (PTA) and acetate kinase (AK), from the butyrate-forming branch, catalyzed by phosphotransbutyrylase (PTB) and butyrate kinase (BK). It is desirable to reduce the production of acetate in the fermentation so that the production of butyrate may be increased accordingly. This is made possible by inactivating the genes in the acetate formation pathway of the bacteria.

Both ack gene, encoding AK, and pta gene, encoding PTA, have been cloned, sequenced, and characterized from several microorganisms, including Escherichia coli, Methanosarcina thermophila, and C. acetobutyricum. Partial DNA sequences of ack and pta genes in C. tyrobutyricum were obtained by PCR Amplification as follows: Synthetic oligonucleotides based on known sequences of cloned ack and pta genes from E. coli, C. acetobutylicum, and B. subtilis, and codon usage preference of C. tyrobutyricum were designed as primers for PCR. The sequences of the PCR primers for ack gene were 5′-GATAC(A/T)GC(A/T)TT(C/T)CA(C/T)CA(A/G)AC-3′ and 5′-(G/C)(A/T)(A/G)TT(C/T)TC(A/T)CC(A/T)AT(A/T)CC(A/T)CC-3′. The sequences of primers for pta gene were 5′-GA(A/G)(C/T)T(A/T/G)AG(A/G)AA(A/G)CA(T/C)AA(A/G)GG(A/T)ATGAC-3′ and 5′-(A/T)GCCTG(A/T)(G/A)C(A/T)GC(A/T/C)GT(A/T)AT(A/T)GC-3′. The amplification using C. tyrobutyricum genomic DNA as the template and designed oligonucleotides as primers was conducted in a DNA engine to obtain the DNA fragments of ack and pta with expected sizes of 560 bp and 730 bp, respectively, which were then cloned into PCR vector pCR 2.1 using TA cloning and sequenced to verify their identity.

Non-replicative plasmids carrying the partial pta or ack gene were then constructed for use in integrational mutagenesis to create the pta or ack knock-out mutants. A 1.5 kb Sph I fragment was removed from plasmids pCR-AK (4.5 kb) and pCR-PTA (4.65 kb) containing the partial genes of ack and pta respectively, and the vectors were relegated to form pCR-AK1 and pCR-PTA1. A 1.6 kb Hind III fragment containing the Em^{r} cassette was removed from pDG 647, and then ligated into Hind III digested pCR-AK1 and pCR-PTA1. The resulting plasmids, pAK-Em (4.6 kb) and pPTA-Em (4.75 kb), were used as integrational plasmids for gene inactivation.

The plasmid DNA (pAK-Em or pPTA-Em) was introduced into C. tyrobutyricum by electroporation, and the mutant cells that had non-replicative plasmids integrated into their chromosome were selected by culturing on agar plates with Em as the selective pressure. Transformation of integrational plasmids into C. tyrobutyricum was carried out using a Bio-Rad Gene pulser in an anaerobic chamber. The competent cells of C. tyrobutyricum were prepared as follows: after overnight growth, a 5 ml culture at late log-growth phase was used to inoculate 40 ml CGM supplied with 40 mM DL-threonine. Cells were grown for 4 h to reach ˜0.8 OD₆₀₀, and then harvested, washed twice and suspended in ice-cold electroporation buffer (SMP; 270 mM sucrose, 7 mM sodium phosphate, pH 7.4, 1 mM MgCl₂). A 0.5 ml of cell suspension was chilled on ice for 5 min in a 0.4 cm electroporation cuvette, and 10˜15 μg of the plasmid DNA (pAK-Em or pPTA-Em) were added to the suspension and mixed well. After the pulse had been applied (2.5 kV, 600Ω, 25 μF), cells were transferred to 5 ml CGM and incubated for 3 h at 37° C. prior to plating on RCM containing 40 μg/ml Em.

Two mutants, PAK-Em and PPTA-Em with inactivated ack and pta, respectively, were obtained and the effects of these mutations on enzyme activities and fermentation kinetics were investigated. Exponential-phase cultures of C. tyrobutyricum wild-type, PAK-Em and PPTA-Em were harvested and cell extracts were assayed for acetate and butyrate-producing enzymes (AK, PTA, BK, PTB). PAK-Em displayed 54% lower AK activity and approximately 130% higher PTA activity than the wild-type. But the activities of butyrate-producing enzymes were approximately the same in PAK-Em and the wild-type. PPTA-Em had only 20˜40% of AK and PTA activities, 135% of BK activity, and similar PTB activity as compared to the wild type.

Batch and fed-batch fermentations with free cells of C. tyrobutyricum wild-type, PAK-Em and PPTA-Em, respectively, were performed in 5 L stirred-tank fermentors in the clostrial growth medium containing glucose (30 g/L) and 40 μg/ml erythromycin (Em) to study the effects of ack/pta knock-out mutations on the fermentation kinetics. Anaerobiosis was reached by initially sparging the medium with nitrogen. The medium pH was adjusted to ˜6.0 with 6 N HCl before inoculation with ˜100 ml of cell suspension prepared in serum bottles. Experiments were carried out at 37° C., 150 rpm, and pH 6.0 controlled by adding either NH₄OH or NaOH. The fed-batch mode was operated by pulse feeding concentrated substrate solution when the sugar level in the fermentation broth was close to zero. The feeding was continued until the fermentation ceased to produce butyrate due to product inhibition. A Micro-oxymax gas analysis system was connected to the fermentor for automatic measurement of gas (H₂ and CO₂) production. Samples from the fermentation broth were analyzed for cell density by optical density (OD) measurement, and glucose and acid product concentrations by using a high performance liquid chromatograph. The results were used to estimate the specific growth rate (μ) and product yields, and are compared in the following table for the wild type and two mutants.

TABLE 1
Kinetics of fed-batch fermentations of glucose with free cells of
C. tyrobutyricum wild-type and mutants at pH 6.0, 37° C.

| | | Wild-type | PAK-Em | PPTA-Em |
|---|---|---|---|---|
| Cell | μ (h⁻¹) | 0.28 ± 0.03 | 0.19 ± 0.02 | 0.19 ± 0.02 |
| | Yield (g/g) | 0.13 | 0.11 | 0.15 |
| Butyrate | Concentration | 28.6 | 43.0 | 42.1 |
| | (g/L) | | | |
| | Yield (g/g) | 0.34 ± 0.01 | 0.47 ± 0.03 | 0.40 ± 0.02 |
| | Productivity | 0.48~0.63 | 0.62~1.35 | 0.61~0.96 |
| | (g/L · h) | | | |
| Acetate | Concentration | 9.7 | 11.9 | 12.2 |
| | (g/L) | | | |
| | Yield (g/g) | 0.12 ± 0.01 | 0.12 ± 0.01 | 0.13 ± 0.01 |
| Butyrate/Acetate ratio (g/g) | | 3.0 | 3.7 | 3.2 |
| Gas | H₂ yield (g/g) | 0.015 | 0.024 | 0.012 |
| | CO₂ yield (g/g) | 0.305 | 0.37 | 0.251 |
| | H₂/CO₂ ratio | 1.08 | 1.44 | 1.06 |
| | (mol/mol) | | | |

As can be seen in Table 1, both mutants grew much slower than the wild-type, but produced more butyric acid to reach a high final concentration of ˜43 g/L, which was ˜50% higher than that obtained in wild-type fermentation. However, inactivating pta or ack gene did not significantly reduce acetate production in the fermentation, suggesting the existence of other enzymes (or pathways) also leading to acetate formation. Nevertheless, both mutants showed a higher selectivity of butyrate production over acetate production as indicated by the increased butyrate to acetate ratio in the fermentation products. The increased butyrate to acetate ratio suggested that ack and pta deletion shifted more carbon flux towards butyrate production, resulting in higher butyrate productivity and yield. The ack deleted mutant PAK-Em increased butyrate yield from 34% in wild-type to 47%. Unexpectedly, the PAK-Em mutant also produced 50% more hydrogen (0.024 g/g) from glucose than the wild type. The pta deleted mutant PPTA-Em also had a higher butyrate yield but a slightly lower hydrogen yield than the wild-type.

Inactivating ack or pta gene by using integrational plasmids did not eliminate or significantly reduce acetate formation even though the corresponding enzyme activity was reduced by more than 50% as compared to the wild type. It is possible that other enzymes such as the butyrate producing enzymes PTB and BK were responsible for some of the acetate formation in mutant C. tyrobutyricum during the fermentation. The specific growth rate of both mutants was reduced by about 32% as compared to the wild-type, indicating that the reduced carbon flux through the acetate production pathway imposed a metabolic burden on cells because of reduced ATP generation per mole of substrate metabolized. The increased carbon flux through the butyrate formation pathway, which produces less ATP per mole of carbon substrate, alleviated the problem but could not make up all the ATP lost in the impaired acetate pathway. Consequently, the mutants grew much slower than the wild type. The reduced cell growth rate could limit the application of these mutants in industrial fermentation for butyric acid production.

Both mutants showed much higher tolerance to butyrate inhibition as indicated by the higher final butyrate concentration, which was ˜50% higher than that from the wild type. The enhanced butyrate tolerance also contributed to the higher butyrate productivity in the fermentation. PTA in C. tyrobutyricum was found to be more strongly inhibited by butyric acid than PTB. It is possible that disrupting the butyrate-sensitive PTA and acetate-forming pathway made the mutants less sensitive to butyrate inhibition because the mutants used mainly the butyrate-forming pathway to generate ATP needed for biosynthesis and maintaining a functional pH gradient across the cell membrane. Although the final butyric acid concentration in the fermentation with mutants can reach ˜43 g/L, the energy cost for product recovery and purification would still be high at this concentration level. In general, it requires a product concentration of at least 50 g/L or higher in order for organic acid fermentations to economically compete with petrochemical synthesis processes.

### Culture Adaptation and Fermentation in Fibrous-Bed Bioreactor

Cells were cultured in a laboratory-scale fibrous bed bioreactor (FBB), which was made of a glass column, and packed with a spiral wound cotton towel. The reactor had a 480 ml working volume, was connected to a 5-L stirred-tank fermentor through a recirculation loop, and was operated under well-mixed condition with pH and temperature controls. Anaerobiosis was maintained by initially sparging the medium in the fermentor with N₂ and then kept the fermentor headspace under 5 psig N₂ during the entire fermentation run. The reactor containing 2 L of medium was maintained at 37° C., agitated at 150 rpm, and pH controlled at 6.0 by adding NH₄OH or NaOH. To start the fermentation, ˜100 ml of cell suspension in serum bottles were inoculated to the fermentor and allowed to grow for 3 days until the cell concentration reached an optical density (OD) of ˜4.0. Cell immobilization was then carried out by circulating the fermentation broth through the fibrous bed at a pumping rate of ˜25 ml/min to allow cells to attach and be immobilized onto the fibrous matrix. After about 36˜48 h of continuous circulation, most of the cells were immobilized and no change in cell density in the medium could be identified. The medium circulation rate was then increased to ˜100 ml/min and the reactor was operated at a repeated batch mode to increase the cell density in the fibrous bed to a stable, high level (>50 g/L). To adapt the culture to tolerate a higher butyrate concentration, the reactor was then operated at fed-batch mode by pulse feeding concentrated substrate solution whenever the sugar level in the fermentation broth was close to zero. The feeding was continued until the fermentation ceased to produce butyrate due to product inhibition. Samples were analyzed for cell, substrate and product concentrations. At the end of the fed-batch experiment, immobilized cells in the FBB were harvested by washing off from the fibrous matrix and stored at 4° C.

Fed-batch fermentation was performed to adapt the bacterial culture to higher butyrate concentrations and to evaluate the maximum butyric acid concentration that can be produced in fermentation. C. tyrobutyricum ATCC 25755 (wild type) was used in this study. Control experiments with free cells were also carried out for comparison purpose. Table 2 compares the fermentation kinetics for butyric acid production from glucose and xylose at pH 6.0 by free cells and immobilized cells respectively. As compared to the free-cell fermentation, the immobilized-cell fermentation in the FBB not only was faster but also produced more butyrate at a much higher final concentration. The highest butyric acid concentration produced in the free-cell fermentation was only ˜19 g/L, whereas butyric acid reached a concentration of ˜40 g/L in the immobilized-cell fermentation. The butyrate yields from glucose and xylose in the immobilized-cell fermentation were also much higher than those in the free-cell fermentations. However, more acetate was also produced in the immobilized cell fermentation. The co-production of acetic acid with butyric acid can complicate the separation and purification of butyric acid from the fermentation broth. It is thus desirable to reduce acetate production in the immobilized cell fermentation in the FBB. Reducing acetate production not only can facilitate the downstream processing but also can increase butyrate production and yield from sugar.

TABLE 2
Kinetics of free-cell and immobilized-cell fermentations of glucose and
xylose by C. tyrobutyricum ATCC 25755 wild-type at pH 6.0, 37° C.

| | Free-cell fermentation | | Immobilized-cell fermentation | |
|---|---|---|---|---|
| Substrate | Glucose | Xylose | Glucose | Xylose |
| Specific growth rate (h⁻¹) | 0.063 ± 0.004 | 0.055 ± 0.004 | 0.094 ± 0.008 | 0.057 ± 0.002 |
| Butyric acid | | | | |
| Final concentration (g/L) | 16.3 | 19.2 | 43.4 | 37.3 |
| Yield (g/g) | 0.34 ± 0.01 | 0.32 ± 0.15 | 0.42 ± 0.02 | 0.41 ± 0.13 |
| Productivity (g/L · h)¹ | 0.19 ± 0.08 | 0.21 ± 0.06 | 6.77 ± 0.23 | 2.2 ± 1.1 |
| Acetic acid | | | | |
| Final concentration (g/L) | 3.6 | 0.7 | 8.4 | 4.9 |
| Yield (g/g) | ~0.06 | ~0.02 | ~0.095 | ~0.085 |

¹Productivity for immobilized-cell fermentation was based on the fibrous bed bioreactor volume of 400 mL, instead of the total liquid medium volume of 2 L in the reactor system.

Butyric acid is known to inhibit cell growth. The specific growth rate for the adapted culture from the FBB was much higher and less sensitive to the butyrate concentration increase as compared with the original culture, indicating a higher tolerance to butyrate. The adapted cells retained more than 50% of its growth ability when the butyrate concentration increased from nil to 30 g/L, whereas the original culture had lost its ability to grow at a butyrate concentration beyond 20 g/L. The effect of butyrate on cell growth can be described by the following noncompetitive product inhibition model:

$$μ = \frac{{μ}_{max} {K}_{P}}{{K}_{P} + P} or \frac{1}{μ} = \frac{1}{{μ}_{max}} + \frac{1}{{μ}_{max} {K}_{P}} P$$

where μ is the specific growth rate (h⁻¹), μₘₐₓ is the maximum growth rate, K_{P} is the inhibition rate constant (g/L), and P is the product (butyric acid) concentration (g/L). The kinetic constants μₘₐₓ and K_{P} can be determined from the linear plot of 1/μ vs. P. Compared to the original culture, the adapted culture not only had a higher μₘₐₓ (0.298 h⁻¹ vs. 0.127 h⁻¹), it also had a much higher K_{P} value (53.89 g/L vs. 1.87 g/L). A 29-fold increase in the K_{P} value clearly indicates that the adapted culture from the FBB is not as sensitive to butyrate inhibition as the original wild type. The increased growth tolerance to product inhibition for cultures adapted in the FBB was also observed in other studies with different microorganisms, including C. formicoaceticum for acetate production and P. acidipropionici for propionic acid production.

The adapted cultures from the FBB are physiologically different from the original cultures used to seed the bioreactor. By immobilization in the fibrous-bed bioreactor (FBB), successfully adapting and selecting an acid-tolerant strain of C. tyrobutyricum that can produce high concentrations of butyrate from glucose and xylose was achieved in a short time period. This mutant grew well under high butyrate concentrations (>30 g/L) and had better fermentative ability as compared to the wild-type strain used to seed the bioreactor. Kinetic analysis of butyrate inhibition on cell growth, acid-forming enzymes, and ATPase activity showed that the adapted cells from the FBB are physiologically different from the original wild type. Compared to the wild type, the adapted culture's maximum specific growth rate increased by 2.3-fold and its growth tolerance to butyrate inhibition increased by 29-fold (based on the K_{P} value). The key enzymes in the butyrate-forming pathway, phosphotransbutyrylase (PTB) and butyrate kinase (BK), were also more active in the mutant, with 175% higher PTB and 146% higher BK activities. Also, the mutant's ATPase was less sensitive to inhibition by butyric acid, as indicated by a 4-fold increase in the inhibition rate constant, and was more resistant to the enzyme inhibitor N,N′-dicyclohexylcarbodiimide (DCCD). The lower ATPase sensitivity to butyrate inhibition might have contributed to the increased growth tolerance to butyrate inhibition, which also might be attributed to the higher percentage of saturated fatty acids in the membrane phospholipids (74% in the mutant vs. 69% in the wild type). This study showed that cell immobilization in the FBB provided an effective means for in-process adaptation and selection of mutants with higher tolerance to inhibitory fermentation products.

However, the final butyric acid concentration produced in the FBB fermentation with C. tyrobutyricum wild-type was still lower than 50 g/L, a threshold level usually needed for economical recovery and purification of organic acids produced in fermentation. The next series of experiments were thus targeted on developing extreme acid tolerant mutants that can be used to economically produce butyrate at concentrations higher than 50 g/L and with high product yields and productivity.

Butyric acid fermentations were carried out with PPTA-Em and PAK-Em mutants immobilized in fibrous bed bioreactors. As can be seen in Tables 3 and 4, butyric acid production from glucose and xylose was significantly improved over the free-cell fermentation, and the final butyric acid concentrations in these fed-batch fermentations reached ˜50 g/L, with the butyrate yield also increased to ˜0.45 g/g. The improved fermentation performance was also better than that obtained with the wild-type in the FBB fermentation, which gave a final butyrate concentration of ˜40 g/L and butyrate yield of ˜0.42 g/g (see Table 2). The relatively low specific growth rates for immobilized cells of PPTA-Em can be attributed to growth inhibition by the high cell density in the FBB environment.

TABLE 3
Kinetics of free-cell and immobilized-cell fermentations of glucose and
xylose by C. tyrobutyricum PPTA-Em at 37° C., pH 6.0.

| | Strains | | | |
|---|---|---|---|---|
| | Free cells | | Immobilized cells | |
| | Sugar Sources | | | |
| | Glucose | Xylose | Glucose | Xylose |
| Cell Growth | | | | |
| μ (h⁻¹) | 0.137 ± 0.032 | 0.086 ± 0.020 | 0.095 ± 0.036 | 0.048 ± 0.006 |
| Biomass yield (g/g) | 0.141 ± 0.024 | 0.109 ± 0.013 | 0.087 ± 0.008 | 0.069 ± 0.003 |
| Acid Production | | | | |
| Butyric acid conc. (g/L) | 37.2 ± 4.8 | 33.5 ± 2.9 | 49.9 ± 0.5 | 51.5 ± 3.5 |
| Butyric acid yield (g/g) | 0.38 ± 0.01 | 0.38 ± 0.02 | 0.44 ± 0.01 | 0.45 ± 0.02 |
| Acetic acid conc. (g/L) | 4.2 ± 0.01 | 3.9 ± 0.3 | 8.7 ± 0.6 | 6.4 ± 1.6 |
| Acetic acid yield (g/g) | 0.058 ± 0.004 | 0.045 ± 0.001 | 0.081 ± 0.005 | 0.045 ± 0.016 |
| B/A ratio (g/g) | 8.9 | 8.6 | 5.7 | 8.0 |
| Gas Production | | | | |
| H₂ yield (g/g) | 0.018 ± 0.001 | 0.017 ± 0.001 | 0.016 ± 0.001 | 0.015 ± 0.001 |
| CO₂ yield (g/g) | 0.389 ± 0.027 | 0.373 ± 0.031 | 0.388 ± 0.005 | 0.348 ± 0.004 |
| H₂/CO₂ ratio (mole/mole) | 1.05 ± 0.03 | 0.95 ± 0.06 | 0.93 ± 0.02 | 0.98 ± 0.03 |

TABLE 4
Kinetics of free-cell and immobilized-cell fermentations of glucose and
xylose by C. tyrobutyricum PAK-Em at 37° C., pH 6.0.

| | Strains | | | |
|---|---|---|---|---|
| | Free cells | | Immobilized cells | |
| | Sugar Sources | | | |
| | Glucose | Xylose | Glucose | Xylose |
| Cell Growth | | | | |
| μ (h⁻¹) | 0.14 ± 0.01 | 0.10 ± 0.01 | 0.14 ± 0.01 | 0.11 ± 0.01 |
| Biomass yield (g/g) | 0.06 ± 0.01 | 0.06 ± 0.01 | 0.04 ± 0.008 | 0.04 ± 0.004 |
| Acid Production | | | | |
| Butyric acid conc. (g/L) | 41.65 ± 0.63 | 39.03 ± 3.46 | 50.1 ± 2.4 | 48.6 ± 2.9 |
| Butyric acid yield (g/g) | 0.42 ± 0.01 | 0.42 ± 0.01 | 0.45 ± 0.02 | 0.45 ± 0.01 |
| Acetic acid yield (g/g) | 0.07 ± 0.01 | 0.08 ± 0.01 | 0.08 ± 0.01 | 0.06 ± 0.01 |
| B/A ratio (g/g) | 5.41 ± 0.61 | 5.19 ± 0.40 | 6.0 ± 0.6 | 8.0 ± 1.6 |
| Gas Production | | | | |
| H₂ yield (g/g) | 0.024 ± 0.001 | 0.024 ± 0.001 | 0.023 ± 0.004 | 0.026 ± 0.001 |
| CO₂ yield (g/g) | 0.37 ± 0.02 | 0.37 ± 0.01 | 0.34 ± 0.01 | 0.35 ± 0.01 |
| H₂/CO₂ ratio (mole/mole) | 1.44 ± 0.06 | 1.44 ± 0.07 | 1.59 ± 0.01 | 1.61 ± 0.01 |
| Carbon Balance (%) | 0.95 ± 0.01 | 0.95 ± 0.06 | 0.96 ± 0.02 | 0.98 ± 0.03 |

In addition to increased butyric acid production, more hydrogen production was also observed in all fermentations with the ack-deleted C. tyrobutyricum mutant, PAK-Em, which showed a much higher hydrogen production as compared with those with the wild type and PPTA-Em. It is not known why this mutant can also produce more hydrogen, which is a valuable biofuel. The potential of producing both butyric acid and hydrogen from various sugar sources by fermentation with PAK-Em was further evaluated at various pH values and the results are summarized in Table 5.

As expected, butyric acid production from glucose was significantly affected by the pH; butyric acid production was 50.1 g/L at pH 5.0, 61.5 g/L at pH 7.0, and only 14.8 g/L at pH 5.0. The low butyric acid production at pH 5.0 was caused by the much stronger butyric acid inhibition at the low pH value. The higher butyric acid concentration at pH 7.0 than at pH 6.0 is also within expectation as more butyric acid would be present in the dissociated form, which is less inhibitory to cell growth, at the higher pH. Surprisingly, a very high butyric acid concentration of 80.2 g/L was reached when the fermentation pH was increased slightly from 6.0 to 6.3 (see FIG. 4). Normally, one would not expect this large improvement with such a small difference in pH. However, the pH and higher butyrate concentration did not significantly affect the product yields, about 0.44±0.02 g/g for butyric acid and 0.07±0.01 g/g for acetic acid at all pH values studied. Hydrogen production appeared to increase slightly with increasing the pH, with the hydrogen yield ranged from 0.022 g/g at pH 5.0 to 0.027 g/g at pH 7.0. The observed lower CO₂ production at higher pH values is attributed to the higher CO₂ solubility in the media with higher pH values.

TABLE 5
Kinetic data of immobilized-cell fermentations in the FBB by C. tyrobutyricum
mutant PAK-Em grown on glucose at 37° C. and various pH values.

| | pH Value | | | |
|---|---|---|---|---|
| | 5.0 | 6.0 | 6.3 | 7.0 |
| Cell Growth | | | | |
| □ (h⁻¹) | 0.08 ± 0.04 | 0.14 ± 0.01 | 0.06 ± 0.02 | 0.09 ± 0.01 |
| Biomass yield (g/g) | 0.11 ± 0.03 | 0.04 ± 0.01 | 0.06 ± 0.01 | 0.07 ± 0.01 |
| Acid Production | | | | |
| Butyric acid conc. (g/L) | 14.79 ± 0.99 | 50.11 ± 2.42 | 80.15 ± 2.54 | 61.52 ± 0.78 |
| Butyric acid yield (g/g) | 0.42 ± 0.03 | 0.45 ± 0.02 | 0.44 ± 0.01 | 0.44 ± 0.01 |
| Acetic acid yield (g/g) | 0.06 ± 0.004 | 0.08 ± 0.01 | 0.07 ± 0.01 | 0.06 ± 0.01 |
| B/A ratio (g/g) | 8.4 ± 5.6 | 6.0 ± 0.6 | 6.3 ± 1.5 | 10.2 ± 1.8 |
| Gas Production | | | | |
| H₂ yield (g/g) | 0.022 ± 0.002 | 0.025 ± 0.001 | 0.026 ± 0.001 | 0.027 ± 0.002 |
| CO₂ yield (g/g) | 0.34 ± 0.04 | 0.34 ± 0.01 | 0.32 ± 0.03 | 0.30 ± 0.01 |
| H₂/CO₂ ratio (mole/mole) | 1.53 ± 0.18 | 1.59 ± 0.01 | 1.85 ± 0.09 | 2.02 ± 0.20 |
| Carbon Balance | 0.96 ± 0.05 | 0.96 ± 0.02 | 0.93 ± 0.03 | 0.94 ± 0.01 |

However, pH does have a significant effect on the production of various metabolites in the FBB fermentation with xylose as the substrate. At pH 5.0, only butyric acid was produced by the PAK-Em mutant, whereas the wild type produced large amounts of acetate (0.43 g/g xylose) and lactate (0.61 g/g xylose) and little butyrate (0.05 g/g xylose). This result indicated that the PAK-Em would be a good butyrate producer even at a low pH value of 5.0, whereas the wild-type would not as there would be a dramatic metabolic pathway shift away from butyrate production caused by the low pH.

TABLE 6
Immobilized cell fermentation of glucose and xylose by C. tyrobutyricum
wild type and PAK-Em at 37° C. and pH 5.0.

| | Wild type | | PAK-Em | |
|---|---|---|---|---|
| | Glucose | Xylose | Glucose | Xylose |
| Cell Growth | | | | |
| Specific growth rate (h⁻¹) | na | 0.04 ± 0.006 | 0.08 ± 0.004 | 0.05 ± 0.01 |
| Biomass yield (g/g)* | na | na | 0.11 ± 0.03 | 0.04 ± 0.003 |
| Final Acid concentration | | | | |
| Butyric acid (g/L) | 14.4 | 5.3 | 14.8 ± 1.0 | 13.6 ± 1.2 |
| Acetic acid (g/L) | 5.43 | 25.5 | 2.2 ± 1.3 | <0.1 |
| Lactic acid (g/L) | 0 | 33.5 | 0 | 0 |
| Product yield* | | | | |
| Butyric yield (g/g) | 0.365 | 0.05 ± 0.01 | 0.42 ± 0.03 | 0.44 ± 0.01 |
| Acetic yield (g/g) | 0.14 | 0.43 ± 0.03 | 0.06 ± 0.004 | 0 |
| Lactic yield (g/g) | 0 | 0.61 ± 0.03 | 0 | 0 |
| Hydrogen (g/g) | na | na | 0.023 ± 0.001 | 0.021 ± 0.001 |
| Carbon dioxide (g/g) | na | na | 0.037 ± 0.007 | 0.30 ± 0.002 |

na: data not available

As discussed earlier, one negative effect of the mutation with ack or pta gene knock-out is the significantly reduced cell growth rate due to the reduced ATP generation per mole of substrate caused by the impaired acetate formation pathway. However, after culturing the PAK-Em mutant in the FBB for an extended period, one adapted mutant (HydEm) isolated from the FBB showed a comparable specific growth rate to the wild type but still can produce much more butyrate and hydrogen than did the wild type and its parental strain PAK-Em (see Table 7). The procedure to obtain this HydEm mutant is as follows. The PAK-Em cells in the FBB were collected after adaptation through six fed-batch fermentations. The adapted cells were washed off from the fibrous matrix under a high pressure and then plated on agar plates to isolate the colonies with higher growth rates. One adapted mutant, HydEm, was selected and characterized in the free-cell fermentation at pH 6.0 and 37° C. with glucose as the substrate. As can be seen in Table 7, the specific growth rate of this mutant (0.21 h⁻¹) was much faster than that of the parent strain PAK-Em (0.14 h⁻¹) and similar to that of the wild type. The cell density in the free-cell fermentation of HydEm (OD₆₀₀=11.5) was much higher than that of PAK-Em (OD₆₀₀=4.4) and the wild type (OD₆₀₀=7.1). The biomass yield of HydEm (0.14 g/g) was also higher than that of the wild type (0.10 g/g) and PAK-Em (0.064 g/g). In addition, the hydrogen production by this mutant was also improved significantly, with a much higher hydrogen yield (0.04 g/g vs. 0.24 g/g) and higher H₂/CO₂ ratio (2.69 vs. 1.44). This result can not be predicted from current knowledge and suggests that the increased hydrogen production and cell growth may be tied together.

TABLE 7
Kinetic of free-cell fermentations of glucose by C. tyrobutyricum
wild-type, PAK-Em, and adapted mutant HydeEm at 37° C., pH 6.0.

| | Strains | | |
|---|---|---|---|
| | Wild type | PAK-Em | HydEm |
| Cell Growth | | | |
| μ (h⁻¹) | 0.21 ± 0.03 | 0.14 ± 0.01 | 0.21 ± 0.02 |
| Biomass yield (g/g) | 0.10 ± 0.01 | 0.06 ± 0.01 | 0.14 ± 0.07 |
| Acid Production | | | |
| Butyric acid conc. | 19.98 ± 3.07 | 41.65 ± 0.63 | 41.38 ± 0.24 |
| (g/L) | | | |
| Butyric acid yield | 0.34 ± 0.02 | 0.42 ± 0.01 | 0.40 ± 0.01 |
| (g/g) | | | |
| Acetic acid yield (g/g) | 0.07 ± 0.01 | 0.07 ± 0.01 | 0.11 ± 0.01 |
| B/A ratio (g/g) | 4.52 ± 0.85 | 5.41 ± 0.61 | 4.01 ± 0.64 |
| Gas Production | | | |
| H₂ yield (g/g) | 0.016 ± 0.001 | 0.024 ± 0.001 | 0.040 ± 0.005 |
| CO₂ yield (g/g) | 0.32 ± 0.02 | 0.37 ± 0.02 | 0.34 ± 0.01 |
| H₂/CO₂ ratio | 1.04 ± 0.01 | 1.44 ± 0.06 | 2.69 ± 0.27 |
| (mole/mole) | | | |
| Carbon Balance | 0.95 ± 0.04 | 0.95 ± 0.01 | 0.93 ± 0.02 |

The disruption of ack gene in PAK-Em reduced the carbon flux through PAT-AK pathway and cells were thus forced to increase hydrogen production in order to recoup some of the lost energy production and to balance the redox potential. Therefore, the ack-deletion mutant would have to produce more hydrogen in order to maintain a good growth rate, which corroborated well with the observed higher growth rate and hydrogen production from the FBB adapted mutant HydEm. This HydEm mutant has the greatest potential of producing butyrate and hydrogen from biomass because of its relatively fast growth rate and high product yields and acid tolerance. Clearly, fermentation using metabolically engineered and environmentally adapted C. tyrobutyricum mutant strains would have the advantages over wild-type and mutant strains derived from merely genetic engineering or environmental adaptation without a proper sequential combination of the two. These experiments clearly showed that rational metabolic engineering of target genes may yield some positive results, but often also led to unexpected results due to the unclear and complex pathways involved in the fermentation.

In summary, Clostridium tyrobutyricum mutants PAK-Em and PPTA-Em with inactivated ack and pta genes, encoding acetate kinase and phosphotransacetylase, respectively, were obtained from integrational mutagenesis and can be used in FBB fermentations to produce high-concentration and high-yield of butyric acid and hydrogen from sugars, including glucose and xylose. The PAK-Em mutant is the better one with higher hydrogen production, and an adapted mutant (HydEm) of this strain showed a good growth rate under free-cell fermentation conditions. FBB fermentation with PAK-Em at pH 6.3 attained the highest butyric acid concentration ever reported for butyric acid fermentation. The previous best results were 62.8 g/L from sucrose, 57.9 g/L from xylose, and 50.1 g/L from glucose. The higher butyric acid concentration from the fermentation would significantly reduce the product recovery and purification costs, which usually account for more than 50% of the total production cost.

Although all previous fermentation experiments used either pure glucose or xylose as the substrate, sugars present in food processing wastes and lignocellulosic hydrolysates can also be readily converted to butyric acid and hydrogen by using the disclosed fermentation method. For example, the sugars (glucose and fructose) present in the waste grape juice from wine manufacturing can be used as the fermentation substrate, and free-cell fermentations at pH 6.0 and 37° C. produced ˜40 g/L of butyric acid with high yields of butyric acid (0.42 g/g) and hydrogen (˜0.024 g/g) from these sugars (see FIG. 5).

### Propionic Acid Fermentation

Propionic acid fermentation was carried out with cells immobilized in a fibrous-bed bioreactor (FBB) connected to a 5-L fermentor through a recirculation loop and operated under well-mixed conditions with pH and temperature controls. The FBB was constructed by packing a spiral wound cotton towel into a glass column bioreactor, and had a working volume of ˜690 mL. After inoculation with ˜100 mL of cell suspension (OD₆₀₀ ˜2.0) into the fermentor, cells were grown for 3-4 days to reach an optical density (OD₆₀₀) of ˜3.5. The fermentation broth was then circulated at a flow rate of ˜30 mL/min through the FBB to allow cells to attach and be immobilized in the fibrous matrix. The process continued for 60-72 h until most cells had been immobilized in the FBB. The medium circulation rate was then increased to ˜80 mL/min, and the fermentation was run at a repeated batch mode to obtain a high cell density in the fibrous bed. Fed-batch fermentation with pulse additions of concentrated glucose solution was then performed to study the fermentation kinetics and to evaluate the achievable maximum propionic acid concentration. At the end of the fed-batch fermentation, adapted cells (mutants) in the FBB were removed from the fibrous matrix by vortexing the matrix in sterile distilled water.

Fed-batch fermentations of glucose by P. acidipropionici ATCC 4875 in free-cell suspension culture and immobilized in a fibrous-bed bioreactor (FBB) were studied. The latter produced a much higher propionic acid concentration (˜72 g/L vs. ˜52 g/L), indicating enhanced tolerance to propionic acid inhibition by cells adapted in the FBB. Compared to the free-cell fermentation, the FBB culture produced 20%-59% more propionate (0.40-0.65 g/g vs. 0.41 g/g), 17% less acetate (0.10 g/g vs. 0.12 g/g), and 50% less succinate (0.09 g/g vs. 0.18 g/g) from glucose. The higher propionate production in the FBB was attributed to mutations in two key enzymes, oxaloacetate transcarboxylase and propionyl CoA:succinyl CoA transferase, leading to the production of propionic acid from pyruvate. Both showed higher specific activity and lower sensitivity to propionic acid inhibition in the mutant than in the wild type. In contrast, the activity of PEP carboxylase, which converts PEP directly to oxaloacetate and leads to the production of succinate from glucose, was generally lower in the mutant than in the wild type. For phosphotransacetylase and acetate kinase in the acetate formation pathway, however, there was no significant difference between the mutant and the wild type. In addition, the mutant had a striking change in its morphology. With a three-fold increase in its length and ˜24% decrease in its diameter, the mutant cell had a ˜10% higher specific surface area that should have made the mutant more efficient in transporting substrates and metabolites across the cell membrane. A slightly lower membrane-bound ATPase activity found in the mutant also indicated that the mutant might have a more efficient proton pump to allow it to better tolerate propionic acid. In addition, the mutant had more longer-chain saturated fatty acids (C17:0) and less unsaturated fatty acids (C18:1), both of which could decrease membrane fluidity and thus might have also contributed to the increased propionate tolerance. The enhanced propionic acid production from glucose by P. acidipropionici was thus attributed to both a high viable cell density maintained in the reactor and favorable mutations resulted from adaptation by cell immobilization in the FBB.

Propionibacterium acidipropionici produces propionic acid from glucose with acetic acid, succinic acid, and CO₂ as byproducts. Inactivation of ack gene, encoding acetate kinase (AK), by gene disruption and integrational mutagenesis was used as a method to reduce acetate formation in propionic acid fermentation. The partial ack gene of ˜750 bp in P. acidipropionici was cloned using a PCR-based method with degenerate primers and sequenced. The deduced amino acid sequence had 88% similarity and 76% identity with the amino acid sequence of AK from Bacillus subtilis. The partial ack gene was used to construct a linear DNA fragment with an inserted tetracycline resistance cassette and a nonreplicative integrational plasmid containing a tetracycline resistance gene cassette, as follows.

The linear DNA fragment with a disrupted ack gene was constructed by inserting a tetracycline resistance (Tet^{r}) cassette into the middle of the partial ack sequence obtained from P. acidipropionici. The 750-bp ack fragment was first cloned into the pNoTA/T7 (cloning vector, 2.7 kb) to obtain pACK (3.5 kb). The Tet^{r} cassette was then inserted at the XmalII site whose sticky end was complementary to that of the NotI, which was the site used to release the Tet^{r} cassette from the pBEST309. The pACK-Tet (5.4 kb) was obtained by ligation of the Tet^{r} cassette and the XmalII-cut pACK plasmid containing the partial ack fragment. The insertion of the Tet^{r} cassette resulted in 400-bp and 350-bp ack fragments located at each end of the inserted cassette. No commonly used restriction enzyme could be used to release the intact ACK-Tet sequence from the pACK-Tet, which had two EcoRI sites located at the ends of the intact ACK-Tet sequence and one EcoRI site located inside the intact. Therefore, partial digestion with EcoRI was carried out to knock off the internal EcoRI site, which was done by removing a few DNA base pairs from the internal restriction site and religate the DNA fragments. This modification occurred at the location near the end of the Tet^{r} cassette and did not show any adverse effect on the gene function. The resulting plasmid pLAT4-dE13 (5.4 kb) was then digested with EcoRI to release the linear ACK-Tet DNA fragment with a size of ˜2.7 kb. The linear ACK-Tet was then used to transform P. acidipropionici to obtain double-cross mutants.

The construction of the nonreplicative integrational plasmid pTAT containing partial ack gene and the Tet^{r} cassette was done as follows. The partial ack gene of ˜500 bp (from the base of 257 to the base of 749) was obtained by BamHI digestion of the 750-bp ack fragment, which had one BamHI restriction site at the base of 257 and another site near the end of the 750-bp fragment. To create the blunt ends with A′-overhang for ligation into pCR®2.1-TOPO® (Invitrogen), both ends of the 500-bp partial ack fragment were first filled with dNTPs by Klenow to create blunt ends, then dephosphorylized by shrimp alkaline phosphatase (GIBCO/BRL) to remove the phosphate group at the 5′-end, and finally added with dATPs by Taq DNA polymerase (Amersham Biosciences) to create A′-overhang at the two blunt ends. The modified partial ack fragment was then cloned into the pCR®2.1-TOPO® (3.9 kb) to obtain the pTOPOACK1 (4.4 kb). The 2.1-kb Tet^{r} cassette, obtained from the pDG1515 by digestion with Xbal and Apal, was ligated into Xbal-Apal-digested pTOPOACK1. The resulting integrational plasmid pTAT (6.5 kb) was then used to transform P. acidipropionici.

After introducing these DNA constructs into P. acidipropionici by electroporation, two mutants, ACK-Tet and TAT-ACK-Tet, were obtained. Southern hybridization confirmed that the ack gene in the mutant ACK-Tet was disrupted by the inserted tetracycline resistance gene. As compared to the wild type, the activities of AK were reduced by 26% and 43% in ACK-Tet and TAT-ACK-Tet mutants, respectively. The specific growth rate of these mutants was reduced by ˜25% to 0.10 h⁻¹ (0.13 ⁻¹ for the wild type), probably because of reduced acetate and ATP production. Both mutants produced ˜14% less acetate from glucose. Although ack disruption alone did not completely eliminate acetate production, the propionate yield was increased by ˜13%.

Genetic manipulations performed on the metabolic pathway affected the production of propionic acid and acetic acid in P. acidipropionici. Compared to the non-engineered strains, mutants (ACK-Tet and TAT-ACK-Tet) with partially inactivated ack gene, which is associated with the acetate formation pathway, grew slower in batch fermentation but produced more propionic acid and less acetic acid from sugars. Attempt was thus made to adapt ACK-Tet mutant in the FBB system to obtain an even higher final concentration of propionic acid and an extreme propionic acid-tolerant mutant strain. As shown in FIG. 6, after 6-month continuous fermentation with gradually increasing propionate concentration in the bioreactor, the final propionic acid concentration in the fermentation broth reached 104 g/L, which was 43% higher than the highest concentration (˜72 g/L) previously achieved with the wild-type strain in the FBB. The much higher proionic acid production in this fermentation was a result of increased tolerance to propionic acid by the mutant strain adapted in the FBB, which increased by more than 10 times (see FIG. 7 and Table 8).

FIG. 7 compares the specific growth rates of the wild type, adapted wild type from FBB, ACK-Tet, and adapted ACK-Tet from FBB at various initial propionic acid concentrations (0-20 g/L). As can be seen in this figure, the specific growth rate decreased rapidly in the presence of propionic acid as it increased to 20 g/L, a result of strong inhibition of propionic acid to the cell. However, the adapted ACK-Tet was much less sensitive to propionic acid inhibition. At 20 g/L of propone acid, the adapted ACK-Tet retained about 75% of its specific growth rate at 0 g/L of propionic acid. In contrast, the wild type lost more than 80% of its growth rate under the same condition. Both the unadapted ACK-Tet and FBB-adapted wild type strains had better tolerance to propionic acid than the unadapted wild type. However, the improvement was not as significant as that achieved with the adapted ACK-Tet strain.

TABLE 8
Comparison of inhibition rate constant and maximum specific
growth rate in the non-competitive inhibition equation for
Propionibacterium acidtpropionici wild type,
ACK-Tet, and their FBB-adapted strains.

| Rate | | | | Adapted Ack- |
|---|---|---|---|---|
| constant | Wild type | Ack-Tet | Adapted wild-type | Tet |
| K_{P} (g/L) | 3.84 | 4.88 | 8.93 | 59.53 |
| μₘₐₓ (h⁻¹) | 0.225 | 0.216 | 0.194 | 0.252 |

Compared to the wild type and ACK-Tet, the adapted ACK-Tet strain only had a slightly higher μₘₐₓ (0.25 vs. 0.22 h⁻¹). However, the inhibition rate constant K_{P} was more than 12 times higher for this adapted mutant as compared to its parental strain (59.5 vs. 4.9 g/L). Clearly, the ACK-Tet adapted in the FBB acquired a much higher tolerance to propionic acid. ACK-Tet has only a slightly higher K_{P} value than that of the wild type (4.9 vs. 3.8 g/L); however, the adapted ACK-Tet has a much higher K_{P} value than that of the adapted wild-type (59.5 vs. 8.9 g/L). Not only the knock-out of ack gene from the chromosome of P. acidipropionic affected the acetate production and decreased its sensitivity to propionic acid, it also gave the mutant better adaptability to acquire an extremely high propionic acid tolerance that was never seen before. This finding is totally out of anyone's expectation. There were notable changes in the adapted mutant cells isolated from the FBB. The protein expression level, H⁺-ATPase activity and morphology of the FBB-adapted ACK-Tet mutant were all significantly different from those of the parental ACK-Tet strain. The adapted mutant acquired a slimmer and elongated rod shape and had a higher expression level of H⁺-ATPase, which plays a key role in maintaining the pH or H⁺ gradient across the cell membrane.

In summary, propionic acid fermentation with the ACK-Tet mutant immobilized in the fibrous bed bioreactor gave the maximum theoretical propionic acid yield of ˜0.56 g/g glucose and reached the highest propionic acid concentration of ˜104 g/L ever achieved. The ACK-Tet mutant after adaptation in the FBB is particularly suitable for industrial production of propionic acid from sugars (including glucose, fructose, xylose, lactose, maltose, and sucrose) and other carbon sources (including lactate and glycerol). As shown in FIG. 8, using glycerol as the carbon source for propionic acid production by metabolically engineered P. acidipropionici (ACK-Tet) in the fibrous bed bioreactor resulted in a high propionic acid yield of 0.71 g/g glycerol, which is much higher than that from glucose. In addition, the production of acetic acid in glycerol fermentation was only 0.03 g/g glycerol, much less than that from glucose (0.1 g/g glucose). Thus, a high purity propionic acid, with the propionic acid to acetic acid ratio of 20 (vs. ˜5 from glucose fermentation), can be produced from glycerol fermentation. The highest propionic acid concentration obtained from glycerol fermentation was ˜106 g/L or 2.5 times of the maximum concentration (˜42 g/L) previously reported with glycerol as the substrate in the literature.

1. A fermentation process for the production of organic acids comprising, providing metabolically engineered mutant clostridia or propionic acid bacteria which have had one or both of ack and pta genes disrupted, adapting the mutant bacteria to increase their resistance to acids and to increase their growth rate by immobilizing the mutant bacteria while exposing the mutant bacteria to a fermentable substrate to produce adapted mutant bacteria, and further exposing the adapted mutant bacteria to a fermentable substrate for a time sufficient to provide a final organic acid fermentation product concentration of greater than about 50 g/L.

2. A process as claimed in claim 1 wherein said mutant bacteria comprise C. tyrobutyricum PAK-Em and said organic acid fermentation product comprises butyric acid and hydrogen.

3. A process as claimed in claim 2 wherein said fermentable substrate comprises a sugar.

4. A process as claimed in claim 3 in which said sugar is selected from the group consisting of glucose, fructose, xylose, lactose, maltose, sucrose, and mixtures thereof.

5. A process as claimed in claim 1 wherein said mutant bacteria comprise C. tyrobutyricum PPTA-Em and said organic acid fermentation product comprises butyric acid and hydrogen.

6. A process as claimed in claim 5 wherein said fermentable substrate comprises a sugar.

7. A process as claimed in claim 6 in which said sugar is selected from the group consisting of glucose, fructose, xylose, lactose, maltose, sucrose, and mixtures thereof.

8. A process as claimed in claim 1 wherein said mutant bacteria comprise C. tyrobutyricum HydEm and said organic acid fermentation product comprises butyric acid and hydrogen.

9. A process as claimed in claim 8 wherein said fermentable substrate comprises a sugar.

10. A process as claimed in claim 9 in which said sugar is selected from the group consisting of glucose, fructose, xylose, lactose, maltose, sucrose, and mixtures thereof.

11. A process as claimed in claim 1 wherein said mutant bacteria comprise P. acidipropionici ACK-Tet and said organic acid fermentation product comprises propionic acid.

12. A process as claimed in claim 11 wherein said fermentable substrate comprises a sugar.

13. A process as claimed in claim 11 wherein said fermentable substrate comprises a carbon source selected from lactate and glycerol.

14. A process as claimed in claim 1 wherein said mutant bacteria comprise P. acidipropionici TAT-ACK-Tet and said organic acid fermentation product comprises propionic acid.

15. A process as claimed in claim 14 wherein said fermentable substrate comprises a sugar.

16. A process as claimed in claim 14 wherein said fermentable substrate comprises a carbon source selected from lactate and glycerol.

17. A fermentation process for the production of organic acids comprising, providing metabolically engineered mutant bacteria selected from the group consisting of C. tyrobutyricum, C. butyricum, C. beijerinckii, C. acetobutyricum, C. populeti, and C. thermobutyricum, and P. acidipropionici, which bacteria have had one or both of ack and pta genes disrupted, adapting the mutant bacteria to increase their resistance to acids and to increase their growth rate by immobilizing the mutant bacteria in a fibrous bed reactor while exposing the mutant bacteria to a fermentable substrate to produce adapted mutant bacteria, and further exposing the adapted mutant bacteria to a fermentable substrate for a time sufficient to provide a final organic acid fermentation product concentration of greater than about 50 g/L.

18. A process as claimed in claim 17 wherein said mutant bacteria comprise C. tyrobutyricum PAK-Em and said organic acid fermentation product comprises butyric acid and hydrogen.

19. A process as claimed in claim 18 wherein said fermentable substrate comprises a sugar.

20. A process as claimed in claim 17 wherein said mutant bacteria comprise C. tyrobutyricum HydEm and said organic acid fermentation product comprises butyric acid and hydrogen.

21. A process as claimed in claim 20 wherein said fermentable substrate comprises a sugar.

22. A process as claimed in claim 17 wherein said mutant bacteria comprise C. tyrobutyricum PPTA-Em and said organic acid fermentation product comprises butyric acid and hydrogen.

23. A process as claimed in claim 22 wherein said fermentable substrate comprises a sugar.

24. A process as claimed in claim 17 wherein said mutant bacteria comprise P. acidipropionici ACK-Tet and said organic acid fermentation product comprises propionic acid.

25. A process as claimed in claim 24 wherein said fermentable substrate comprises a sugar.

26. A process as claimed in claim 24 wherein said fermentable substrate comprises glycerol.

27. A process as claimed in claim 17 wherein said mutant bacteria comprise P. acidipropionici TAT-ACK-Tet and said organic acid fermentation product comprises propionic acid.

28. A process as claimed in claim 27 wherein said fermentable substrate comprises a sugar.

29. A process as claimed in claim 27 wherein said fermentable substrate comprises glycerol.

30. A fermentation process for the production of butyric acid comprising, providing metabolically engineered C. tyrobutyricum PAK-Em, C. tyrobutyricum HydEm, or C. tyrobutyricum PPTA-Em mutant bacteria which have had one or both of ack and pta genes disrupted by homologous recombination, adapting the mutant bacteria to increase their resistance to acids and to increase their growth rate by immobilizing the mutant bacteria in a fibrous bed reactor while exposing the mutant bacteria to a fermentable substrate to produce adapted mutant bacteria, and further exposing the adapted mutant bacteria to a fermentable substrate for a time sufficient to provide a final butyric acid fermentation product concentration of greater than about 50 g/L.

31. A process as claimed in claim 30 wherein said fermentable substrate comprises a sugar.

32. A fermentation process for the production of propionic acid comprising, providing metabolically engineered P. acidipropionici ACK-Tet or P. acidipropionici TAT-ACK-Tet mutant bacteria which have had one or both of ack and pta genes disrupted by homologous recombination, adapting the mutant bacteria to increase their resistance to acids and to increase their growth rate by immobilizing the mutant bacteria in a fibrous bed reactor while exposing the mutant bacteria to a fermentable substrate to produce adapted mutant bacteria, and further exposing the adapted mutant bacteria to a fermentable substrate for a time sufficient to provide a final propionic acid fermentation product concentration of greater than about 50 g/L.

33. A process as claimed in claim 32 wherein said fermentable substrate comprises a sugar.

34. A process as claimed in claim 32 wherein said fermentable substrate comprises glycerol.
